(19) United States
(12) Patent Application Publication
(10) Pub. No.: US 2016/0106723 A1
(43) Pub. Date: Apr. 21, 2016

(54) METHODS TO SENSE AND TREAT SUBCLINICAL SKIN DAMAGE

(71) Applicant: Robert K. Ilowite, Branchburg, NJ (US)

(72) Inventor: Robert K. Ilowite, Branchburg, NJ (US)

(21) Appl. No.: 14/884,926

(22) Filed: Oct. 16, 2015

Related U.S. Application Data
(60) Provisional application No. 62/064,803, filed on Oct. 16, 2014.

(51) Int. Cl. A61K 31/437; A61K 49/00; A61K 9/00

(52) U.S. Cl. CPC A61K 31/437; A61K 9/0014; A61K 49/0006

(57) ABSTRACT

A method of sensing and removing subclinical, precancerous cells from skin wherein the unblemished skin is not being treated for AK, fine lines or clinical wrinkles and is not chronically exposed to sun or UV radiations, the method comprising topically applying to skin of a patient a composition essentially comprising of an TLR7 agonist agent, Imiquimod in an amount effective to induce a cellular immune response in the skin resulting in the sensing and removal of subclinical, atypical keratinocytes by erythema, scaling, crusting, or combinations thereof.

### CROSS-REFERENCE TO RELATED APPLICATIONS

This application claims the benefit of U.S. Provisional Application Ser. No. 62/064,803 filed Oct. 16, 2014, entitled “METHODS TO SENSE AND TREAT SUBCLINICAL SKIN DAMAGE,” which is incorporated herein by reference in its entirety.

### BACKGROUND

1. Field

The present invention relates to methods to find and treat subclinical skin damage prior to its development as Actinic keratosis (AK) by an immune-modulating agent. More specifically, the present invention is directed to the use of a synthetic toll-like receptor 7 (TLR7) agonist e.g., Imiquimod, to find and remove atypical skin cells.

2. Related Art

Recent reports indicate increased rates of sun related skin damage, even in instances of minimal sun exposure. This is partly due to environmental factors, such as thinning of the ozone layer and more ultraviolet (UV) radiation reaching the Earth's surface, and partly to lifestyle factors such as spending most of leisure time outdoors and use of tanning beds. The skin damage rates are highest in countries that have a large fair-skinned population and high sun exposure. Often the damage to skin cells is asymptomatic and goes unnoticed until the damaged skin cells become apparent as Actinic keratosis (AK) or solar keratosis at a later stage.

According to a skin care foundation more than 58 million Americans are affected by AK. AK is a skin pre-cancer and indicates sustained sun damage which could develop into any kind of skin cancer, not just squamous cell carcinomas (SCC). Up to 10 percent of AKs may advance to SCC, and 40-60 percent of SCCs begin as untreated AKs. About 2 to 10 percent of these SCCs spread to internal organs and become life-threatening.

Patients with AK experience impairment to their quality of life in terms of physical symptoms such as itching, burning, tenderness, and dyspigmentation. Psychological impairment has also been reported, impacting on the patients' confidence and their sense of well-being.

Furthermore, direct costs relating to AK treatment are very high. In the year 2004, the costs to treat AK was $1.2 billion, with 92% of these costs due to physician office visits and 5% to prescription drugs. For patients, the burden of dealing with AK can be both personal and financial.

The known techniques for treatment of AK include lesion-directed treatment, which includes cryosurgery (liquid nitrogen), laser therapy, Curettage/excision/shave biopsy, electrodessication or field-directed therapy, which includes ingenol mebutate, topical 5-FU, chemical peeling, diclofenac 3% gel, topical photodynamic therapy using either methyl aminolevulinate or 5-aminolevulinic acid, and Imiquimod all of which are associated with side effects and chances of AK recurrence. Importantly, these techniques are used only in the treatment of symptomatic AK not for the treatment of the subclinical stage of AK.

Therefore, the new target for the treatment of damaged skin cells other than those chronically sun exposed and potentially later manifesting as (AK) is the detection/sensing and clearance of subclinical lesions.

Chronically sun exposed younger individuals, preferably between the ages of twenty (20) and fifty-nine (59) years of age, often experience sun damaged skin in a later stage of life. However, subclinical skin damage may not be apparent to those individuals with minimal to moderate exposure to the sun or UV. Therefore, it is highly desirable that rather than delaying treatment until the onset of AK, there is a need reduce morbidity and scarring by prophylactically treating cell damage at an early stage of life. Therefore, there is a need to treat subclinical skin damage pre-clinically through a field directed therapy which can uniquely detect/sense and clear preclinical skin damages. To meet the requirement of such a therapy there is a need for an agent which can sense subclinical skin damage prior to the appearance as AK or SSC.

### SUMMARY

The primary object of the invention is to provide methods of sensing and treating subclinical damage to the skin of a population having minimal to moderate exposure to sun or UV. The present invention is based, at least in part, on the discovery that a composition comprising Imiquimod can be used to sense, treat the subclinical damage of skin cells and prevent AK.

In an embodiment, the present invention provides for a method of resolving at least a portion of the subclinical skin damage, where the skin has no clinical signs of AK and the patient was not previously treated for AK or nonmelanoma skin cancer.

In an embodiment, the present invention provides for a method of sensing and treating subclinical skin damage of subjects with minimal to moderate sun exposure.

In an embodiment, the present invention provides for a method of treatment of skin which is unblemished and not being treated for fine lines or clinical wrinkles.

Another embodiment of the present invention is to provide a method of suppressing initiation of skin cancer by treating subclinical damage of skin cells at inception.

In an embodiment, the present invention provides for a method of removing precancerous cells from sub-clinically damaged skin of a subject.

In an embodiment of the invention, the detection and treatment of said unblemished skin is carried out at an early stage of life preferable between the ages of twenty (20) to fifty-nine (59) years of age.

In yet another embodiment of the invention, the treatment of said unblemished skin is through a topical composition comprising a TLR7 agonist agent.

Another embodiment of the present invention is to provide a method of inducing a favorable immune response in skin treated for a subclinical damage.

In an embodiment of the invention the composition essentially comprises Imiquimod, and one or more pharmaceutically acceptable carriers or excipients.

Another embodiment of the present invention is to provide a composition including a single active agent, that being, Imiquimod in said composition.

Another embodiment of the present invention is to provide a topical composition in effective dosage form, effective to induce erythema, scaling and/or fine scaling, crusting, or combinations thereof.

Another embodiment of the present invention is to provide a detailed method of treatment comprising topically administering an effective amount of a composition comprising Imiquimod during a cycle of therapy that is in the range of two to four weeks; and observing the skin for the response; wherein the composition is administered once or twice daily during the cycle of therapy, which is repeated at least once on a substantially annual basis.

In an embodiment, there is provided a method of discovering sub-clinically damaged or actinically damaged cells which are otherwise not clinically apparent.

In an embodiment, there is provided a method of revealing sub-clinically damaged skin cells.

Another embodiment of the present invention describes a method to discover precancerous cells present in unblemished skin.

Yet another embodiment, the present invention is directed to a method of preventing actinic keratosis in a subject by selecting a subject who is susceptible to the development of actinic keratosis and administering to said subject an effective dosage of a composition comprising Imiquimod thereby preventing actinic keratosis in said subject. The said subject is preferably in the age group of twenty (20) to fifty-nine (59) years of age.

Another embodiment of the present invention provides a method of managing a healthy skin condition of an individual by undergoing a cycle of therapy that is in the range of two to four weeks which is repeated at least once on a substantially annual basis.

Another embodiment of the present invention provides a method of maintaining unblemished skin using Imiquimod where the skin has no clinical signs of damage, no previous actinic keratosis, and no previous nonmelanoma skin cancer.

While the present invention has identified what it believes to be concentrations of Imiquimod formulations, numbers of applications per week and durations of therapy, it should be understood by those versed in this art that any effective concentration of Imiquimod in a formulation that delivers an effective amount of Imiquimod and any numbers of application per week during a short duration of therapy, as described herein, that can effectively treat sub-clinically damaged skin, without causing treatment limiting local skin reactions or related adverse events, including too many rest periods, is contemplated by the present invention.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures. Optional portions of the figures may be illustrated using dashed or dotted lines, unless the context of usage indicates otherwise.

### BRIEF DESCRIPTION OF THE PHOTOGRAPHS

FIG. 1A is a color photograph of chest region of a female participant before treatment;

FIG. 1B is a color photograph of this participant who showed a reaction of severe erythema after 11 days of treatment, at a strength of about 3.75%;

FIG. 2A is a color photograph of chest region of a female participant before treatment;

FIG. 2B is a color photograph of this participant who showed a reaction of moderate erythema after fourteen days of treatment, at a strength of about 5.00%;

FIG. 3A is a color photograph of chest region of a female participant before treatment;

FIG. 3B is a color photograph of this participant who showed a reaction of moderate erythema after fourteen days of treatment, at a strength of about 3.75%;

FIG. 4A is a color photograph from the cheek region of a male participant before treatment;

FIGS. 4B and 4C are color photographs of this participant who showed a mild reaction after twelve days of treatment, at a strength of about 2.5%;

FIG. 5A is a color photograph from the forehead region of a male participant before treatment;

FIG. 5B is a color photograph of this participant who showed a moderate reaction after fifteen days of treatment, at a strength of about 3.75%;

FIG. 6A is a color photograph from the scalp region of a male participant before treatment;

FIG. 6B is a color photograph of this participant who showed a moderate reaction after ten days of treatment, at a strength of about 2.5%;

FIG. 7A is a color photograph from the cheek region of a male participant before treatment;

FIG. 7B is a color photograph of this participant who showed a moderate reaction after fourteen days of treatment, at a strength of about 2.5%;

FIG. 8A is a color photograph of cheek region of a female participant who showed a moderate reaction after fourteen days of treatment, at a strength of about 2.5%;

FIG. 8B is a color photograph of forehead region of a female participant who showed a moderate reaction after fourteen days of treatment, at a strength of about 2.5%;

FIG. 8C is a color photograph of forehead region of a female participant who showed a moderate reaction after fourteen days of treatment, at a strength of about 2.5%.

### DETAILED DESCRIPTION

In the following detailed description, numerous specific details are set forth in order to provide a thorough understanding of embodiments or other examples described herein. In some instances, well-known methods, procedures and compositions have not been described in detail, so as to not obscure the following description.

Further, the examples disclosed are for exemplary purposes only and other examples may be employed in lieu of, or in combination with, the examples disclosed. It should also be noted the examples presented herein should not be construed as limiting of the scope of embodiments of the present disclosure, as other equally effective examples are possible and likely.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B, and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

As used herein, the term “senses” refers to detection of damaged skin cells. It also refers to detection of a molecular damage to the skin cells. The damage that can be sensed or detected could be at the level of DNA or protein or physiological.

As used herein, the term “molecular stage” refers to a stage when the damage is limited to a biomolecule present in a cell. The biomolecule may be a protein or nucleic acid.

As used herein, the terms “sun or UV radiation damage” refers generally to physiologically damaged skin cells where chronic UV exposure leads to cumulative DNA alterations devastating physiological DNA repair mechanisms. Such a damage can be found most commonly on sun-exposed areas such as the scalp, head, neck, hands or other areas of the body.

As used herein, the terms “subclinical damage” means a condition of skin with no clinical sign of damage, unblemished skin, that is, skin with no clinically apparent AKs or non-melanoma skin cancer, including but not limited to, fine lines or clinical wrinkles.

As used herein, the term “effective amount or effective dose” includes an amount effective, at dosages and for periods of time necessary, to achieve the desired result, e.g., to induce erythema, scaling, crusting, or combinations thereof and resolve at least a portion of the subclinical skin damage. An effective amount is also one in which any toxic or detrimental effects (e.g., side effects) of the Imiquimod are outweighed by the therapeutically beneficial effects.

As used herein, the terms “treat,” “treatment” and “treating” include the topical application of an effective dose of the compositions essentially comprising of Imiquimod, to a subject whose skin contains no clinical signs of AKs.

As used herein, the terms “early stage of life”, refers to individuals in the age group of twenty (20) to fifty-nine (59) years of age.

As used herein, the terms “later stage of life”, refers to individuals in the age group of sixty (60) to ninety (90) years of age.

As used herein, the terms “actinic keratosis” and/or AK refer generally to the art recognized condition, also known as solar keratosis, characterized by thick, scaly, fine scaly or crusty patches of skin, often in the form of lesions or micro-erosions, often surrounded by red, irritated skin. Such conditions can be found most commonly on sun-exposed areas such as the scalp, head, neck, hands or other areas of the body. The condition may be a premalignant disorder and may progress to squamous cell carcinoma. All forms of actinic keratosis are intended to be included by the term “actinic keratosis” including, but not limited to, actinic cheilitis, inflammatory keratosis, hypertrophic keratosis and pigmentary keratosis.

Before describing several exemplary embodiments of the present invention, it is to be understood the presently claimed invention is not limited to the details of construction or process steps set forth in the following description. The presently claimed invention is capable of other embodiments and of being practiced or being carried out in various ways.

The present invention is directed to a method of sensing and removing precancerous cells from the unblemished skin of a subject with minimum to moderate exposure to sun or UV radiations, such as damage that effects the cell at molecular level and is directed towards the biomolecules like protein and nucleic acids present in the cell. Such damage to the cell is limited to the molecular stage and has not reached a clinically detectable form in the cell. The method comprises topically applying to skin of a subject a composition essentially comprising an immunomodulator in an amount effective to induce erythema, scaling, crusting, or combinations thereof.

The present invention is directed to a method of treating the subclinical form of actinic keratosis (AK). The present invention is based, at least in part, on the discovery that a topical composition comprising a TLR7 agonist is capable of treating a subclinical skin condition wherein skin cells are damaged. The said TLR7 agonist induces erythema, scaling, crusting, or combinations thereof and resolve at least a portion of the subclinical sun damage.

Imiquimod is a topical immune response modifier and Toll-like receptor 7 (TLR7) agonist that induces the immunological destruction of sub-clinically damaged skin cells. TLR7 activation by Imiquimod has pleiotropic effects on innate immune cells. Treatment of normal human skin with Imiquimod induced activation of resident T cells and reduced IL-10 production. Thus, Imiquimod stimulates tumor destruction by recruiting cutaneous effector T cells from blood and by inhibiting tonic anti-inflammatory signals within the tumor.

Another mechanism by which Imiquimod destroys sub-clinically damaged cells is by activating macrophages and other cells via binding to surface receptors thereby inducing secretion of pro-inflammatory cytokines. The cytokines generate cytotoxic effectors which then generate their effect on sub-clinically and early damaged skin cells.

Topical application of Imiquimod leads to visualization and treatment of subclinical Actinic Keratoses. In an embodiment of this invention Imiquimod is used in an effective dose to sense and visually expose and, at the same time, also treat clinically invisible subclinical AK. Methods disclosed in this invention help in the early detection and “preventive” treatment of sub-clinically damaged skin, said method is easier, more effective, and less burdensome than a later treatment of clinically evident cancer.

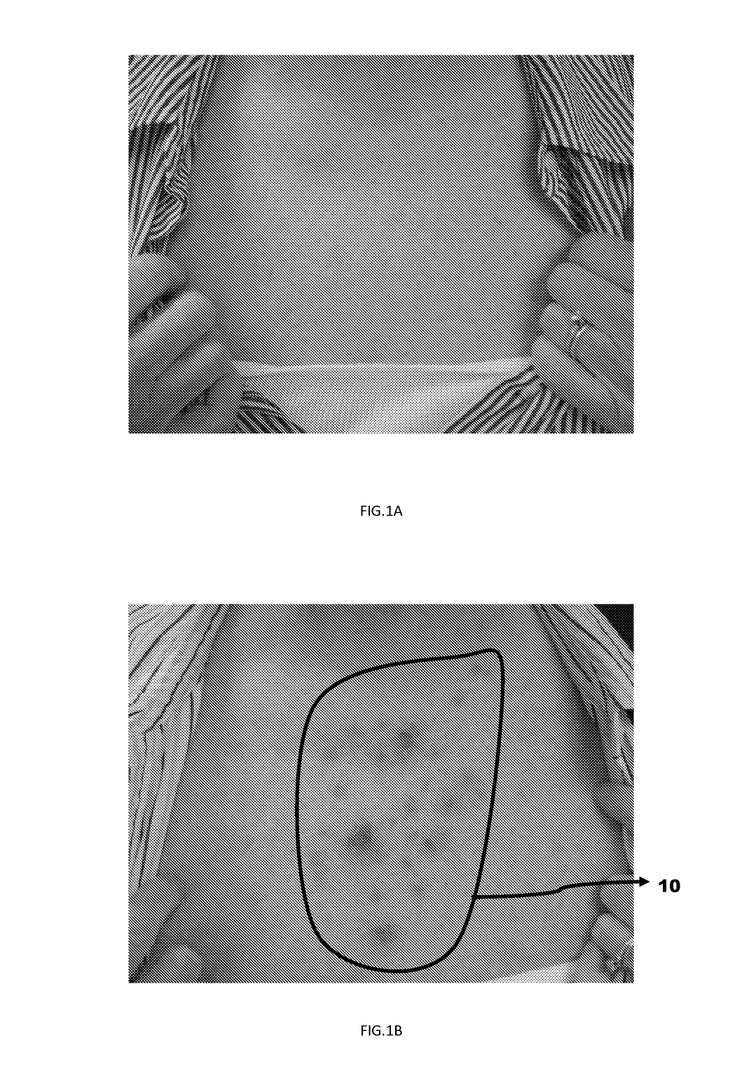

According to an embodiment of the present invention depending on age, lifestyle, and skin type, subclinical skin damage may develop in chronic or minimally sun-exposed skin, predominantly on chest cheek, frontal temporal or forehead areas, and on bald male scalps, neck, hands or other areas of the body FIGS. 1-8.

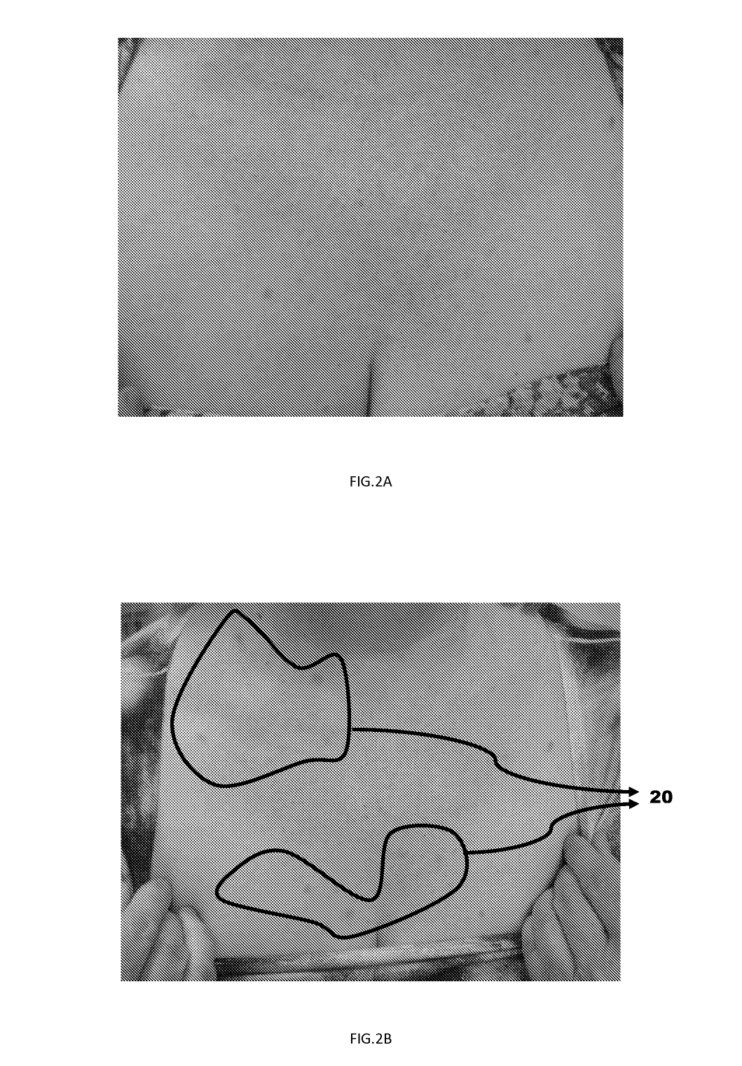

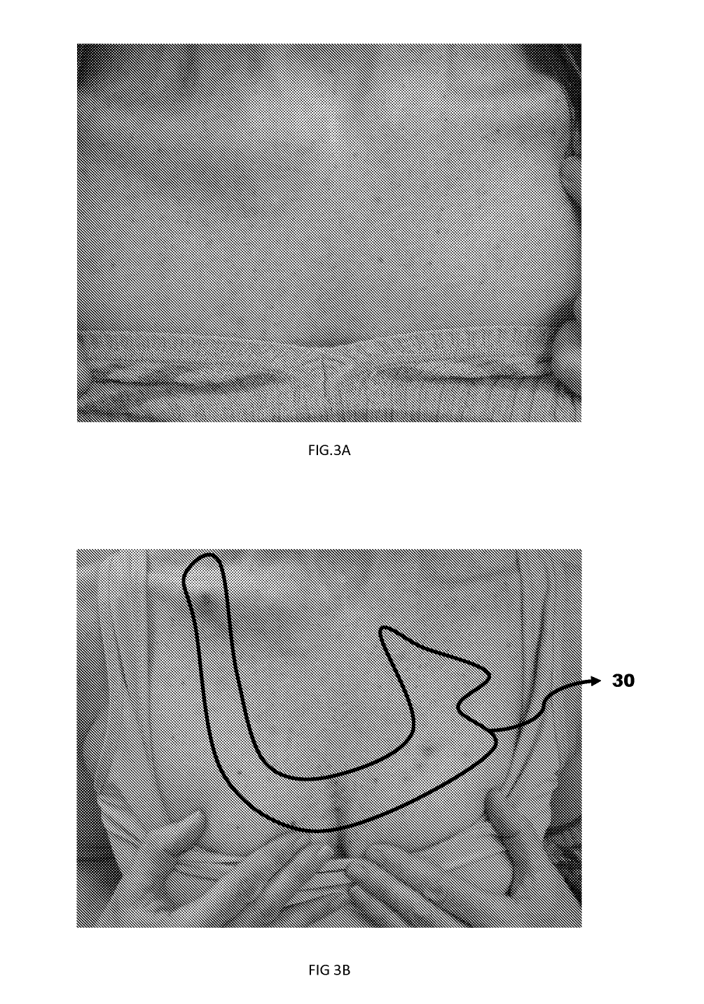

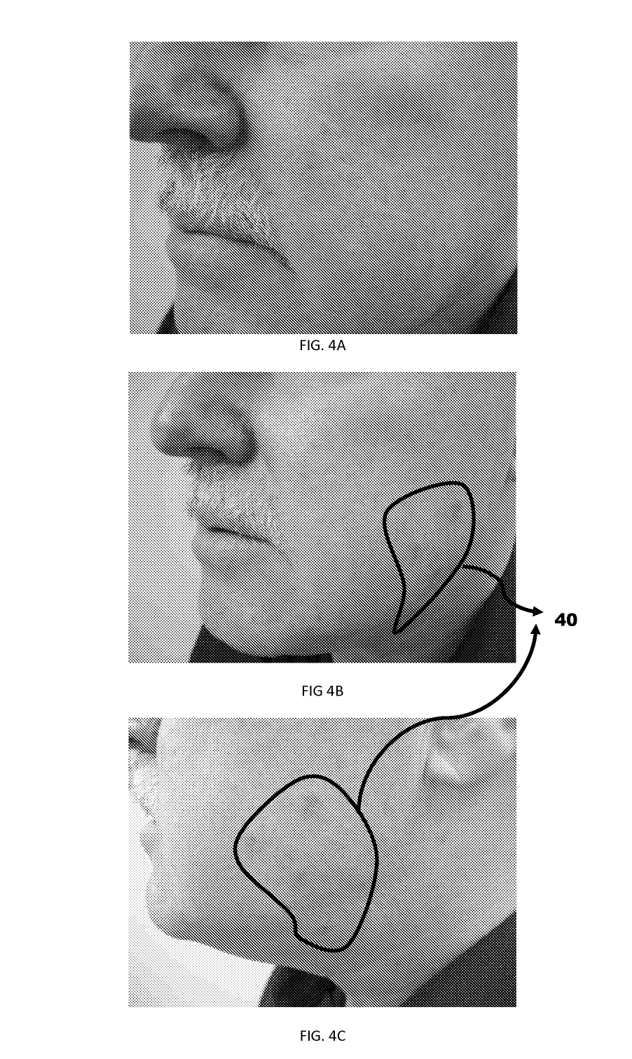

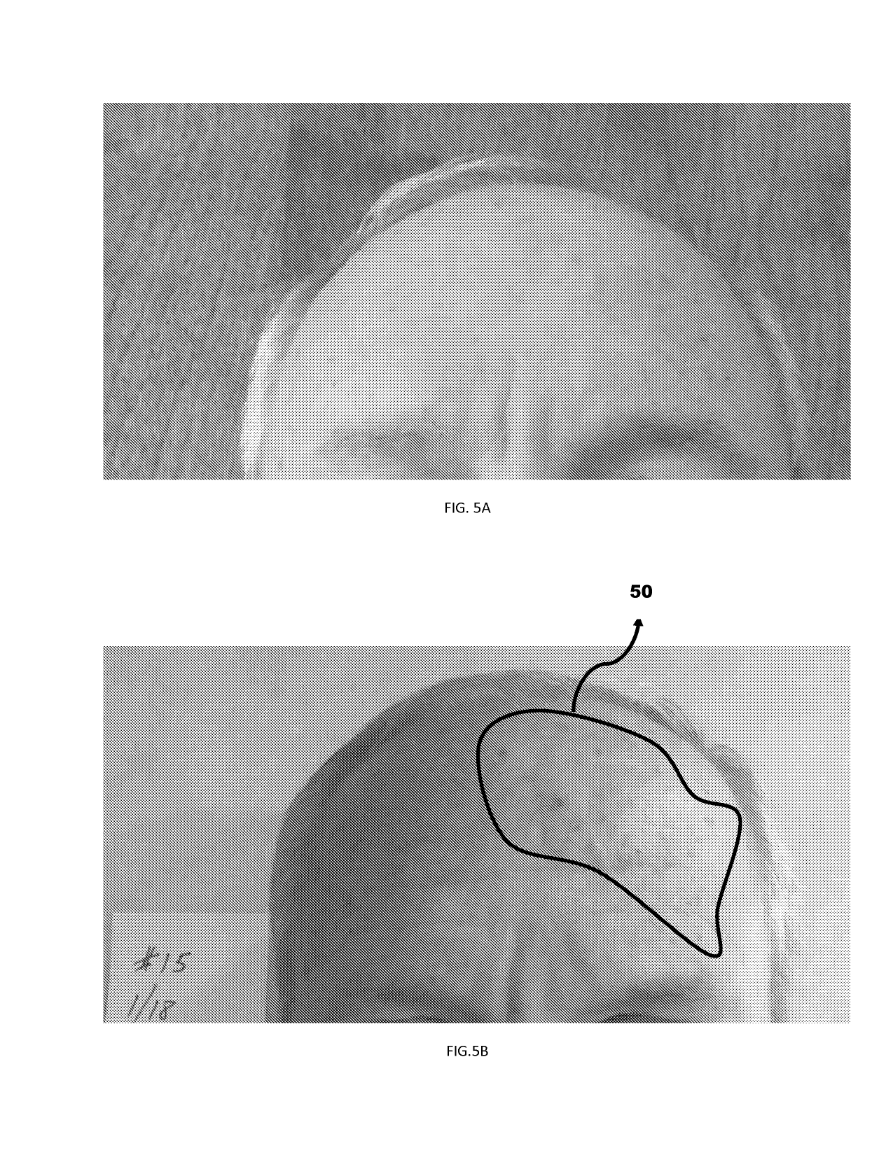

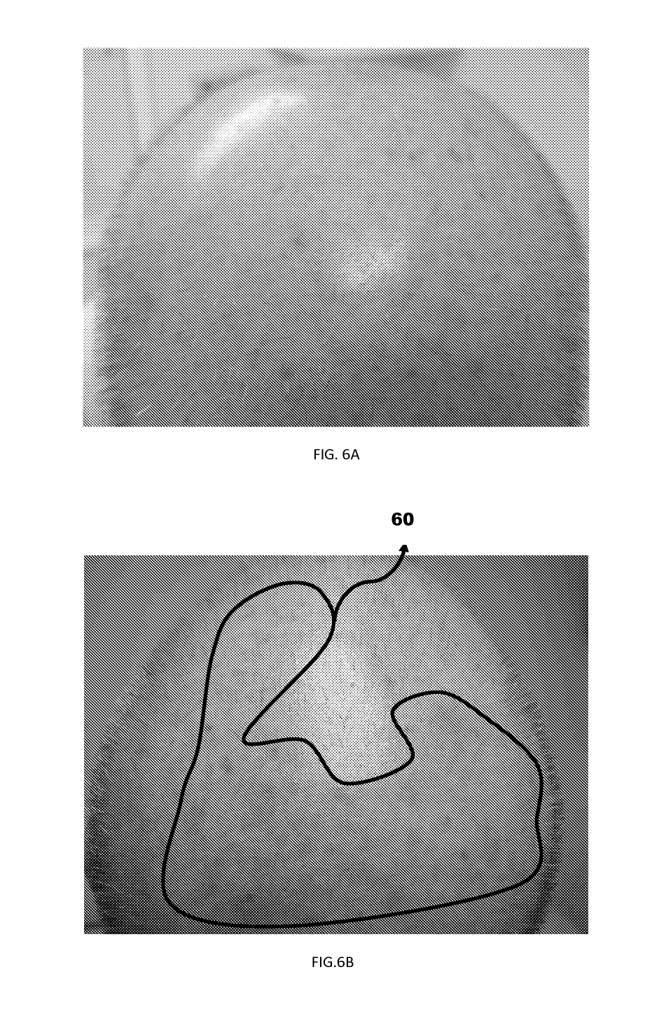

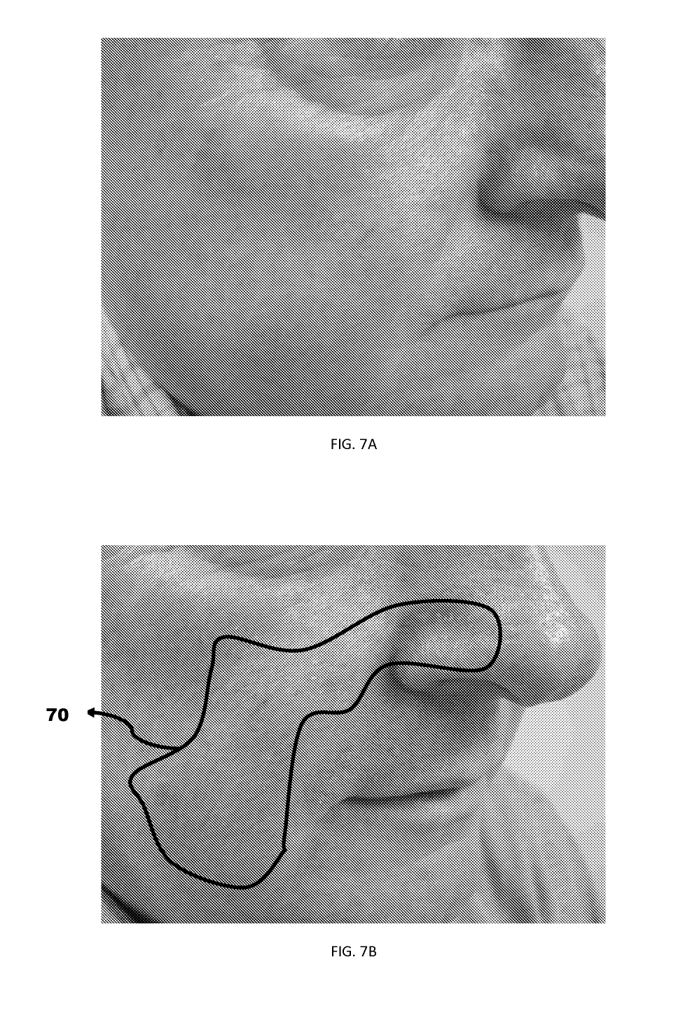

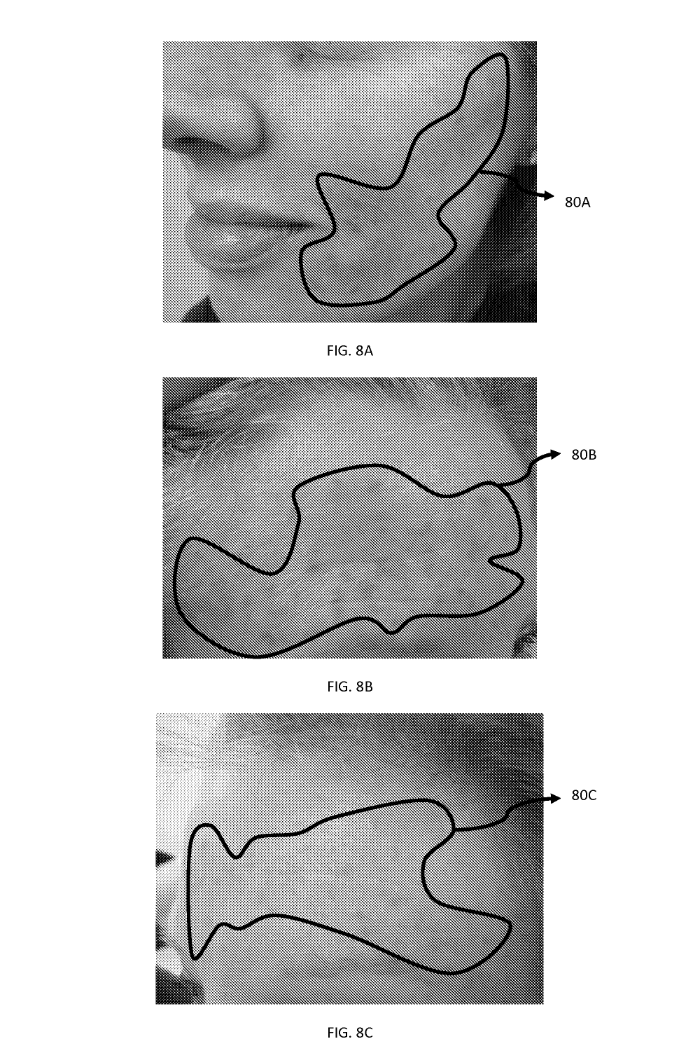

Non-chronic or minimally sun or UV exposed skin at an early stage of life contains subclinical damages and shows very early clinically invisible AKs, which can be revealed visually by triggering an inflammatory reaction in them with Imiquimod FIG. 1B region 10, FIG. 2B region 20, FIG. 3B region 30, FIG. 4B, 4C region 40, FIG. 5B region 50, FIG. 6B region 60, FIG. 7B region 70, FIG. 8 regions 80A, B and C.

Embodiments of the present invention disclose the heretofore unknown use of Imiquimod to invoke a response indicative of damaged skin not being treated for fine lines or clinical wrinkles and that was not previously subjected to treatment for AKs or nonmelanoma cancer. It is unexpected that otherwise seemingly undamaged skin can benefit from an agent that historically was only used to treat apparent sun damage, for example, fine lines or clinical wrinkles.

Topically applied Imiquimod at an effective dosage level shows clinical signs of inflammation. The response of sun or UV damaged cells to Imiquimod in these non-suspicious areas are indicative of altered cells. Clearly subclinical skin damage or AK may exist in the skin but may not be clinically evident in their early development as appearing in FIGS. 1A, 2A, 3A, 4A, 5A, 6A, and 7A.

The detailed method of treatment of subclinical skin damage and of inducing an immune cell response in unblemished skin involves topical application of formulation essentially comprising Imiquimod in an effective amount during a cycle of therapy that is in the range of two to four weeks; and observing the skin for the response; wherein the composition is administered once or twice daily during the cycle of therapy, which is repeated at least once on a substantially annual basis.

Treatment of unblemished skin for subclinical skin damage includes the steps of: topically administering to skin of a patient a composition comprising a TLR7 agonist agent e.g Imiquimod in an amount effective to induce erythema, scaling, crusting, or combinations thereof and resolve at least a portion of the subclinical skin damage, where the skin has no clinical sign of skin damage and the patient was not previously treated for actinic keratosis or nonmelanoma skin cancer.

Imiquimod is an immune-modulator which stimulates the immune response by induction, synthesis and release of cytokines. It is believed that Imiquimod induces the release of the cytokines interferon (IFN) α, interleukin (IL) 1, 6 and 12, and tumor necrosis factor (TNF) a by human peripheral blood mononuclear cells. These cytokines stimulate several other aspects of the innate immune response. In addition, Imiquimod stimulates acquired immunity, in particular the cellular arm which is important for control of viral infections and various tumors. Therefore, it is capable of clearing precancerous cells by inducing erythema, scaling, crusting, or combinations thereof.

Imiquimod cream is not currently indicated for the treatment of unblemished skin, that is, skin with no clinically apparent AKs or nonmelanoma skin cancer, including but not limited to, fine lines or clinical wrinkles. During treatment of AKs with Imiquimod cream, subclinical skin damage often will become clinically apparent through the activation of macrophages and other cells via Imiquimod binding to surface receptors thereby inducing secretion of pro-inflammatory cytokines. The cytokines generate cytotoxic effectors which then generate their effect on sub-clinically and early damaged skin cells.

The composition can comprise the TLR7 agonist agent in an amount in the range of 0.25 to 10% by weight, or 0.5 to 5% by weight, or even or 2.5 to 5% by weight. The composition containing the TLR7 agonist agent can include one or more pharmaceutically acceptable carriers or excipients. Exemplary carriers are, but not limited to deionized water, arachidyl alcohol, behenyl alcohol, arachidyl glucoside, montanov 202, cetearyl alcohol, capric/caprilic triglyceride, isopropyl palmitate, steareth-2, dimethicon, steareth-20, allantoin, propylene glycol, methylisothiazolmone, Caprylic/Capric acids; medium chain triglycerides, PEG 400, PEG 3350, Pluronic F127, Polosamer F127, sodium benzoate, and combinations thereof, propylene glycol, tris (hydroxymethyl) aminomethane, hydroxypropylcellulose, parabens (methyl and/or propyl) and disodium edentate. Other inactive ingredients can include: carbomer 940, dimethicone, glycerin, methyl gluceth-20, methyl methacrylate/glycol dimethacrylate crosspolymer, octyl hydroxy stearate, polyethylene glycol 400, polysorbate 80, sorbitan monooleate, stearic acid, and trolamine.

In one aspect of the invention, at least Imiquimod is applied in a mixture with a dermatologically acceptable carrier or vehicle (e.g., as a lotion, cream, ointment, soap, stick, or the like) so as to facilitate topical application and, in some cases, provide additional therapeutic effects as might be brought about, e.g., by moisturizing of the affected skin.

Carrier for dermatological compositions can consist of a relatively simple solvent or dispersant such as water, it is generally preferred that the carrier comprise a composition more conducive to topical application, and particularly one which will form a film or layer on the skin to which it is applied so as to localize the application and provide some resistance to washing off by immersion in water or by perspiration. Many preparations are known in the art, and include lotions containing oils and/or alcohols and emollients such as hydrocarbon oils and waxes, silicone oils, vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids.

In yet another aspect, the present invention provides for a composition that is suitable for topical application. For example, the composition may be in the form of a gel, liquid or spray. In addition, the composition may be a sustained release composition, for example, for release of the Imiquimod, a pharmaceutically acceptable salt thereof, or the combination of compounds for at least one, two, three, four, five or six weeks. One embodiment provides that the composition is applied in a dosage of 0.01 to 1 mg/cm2.

As to administration frequency, the composition can be administered in a cycle of therapy that is in the range of two to four weeks. In a detailed embodiment, the composition is administered once or twice daily during the cycle of therapy. Another embodiment provides the cycle of therapy is repeated at least once on a substantially annual basis. By substantially annual basis it is meant that administration can take place at about yearly intervals, where, for example, no fewer than 325 days and no more than 400 days have passed between treatments.

A detailed method of treating subclinical skin damage to skin not being treated for AK, fine lines or clinical wrinkles includes the steps of: topically administering to skin a composition consisting essentially of Imiquimod and one or more pharmaceutically acceptable carriers or excipients in an amount effective to induce erythema, scaling, crusting, or combinations thereof and resolve at least a portion of the subclinical sun damage, where the skin has no clinical signs of damage, no previous actinic keratosis, and no previous nonmelanoma skin cancer. In one embodiment, the composition comprises Imiquimod in an amount in the range of 0.25 to 10% by weight, or in a preferred range of 0.5 to 5% by weight.

While the foregoing is directed to embodiments of the present invention, other and further embodiments of the present invention may be devised without departing from the basic scope thereof. It is understood that various embodiments described herein may be utilized in combination with any other embodiment described without departing from the scope contained herein. Further, the foregoing description is not intended to be exhaustive or to limit the invention to the precise form disclosed.

Modifications and variations are possible in light of the above teachings or may be acquired from practice of the invention. Certain exemplary embodiments may be identified by use of an open-ended list that includes wording to indicate the list items are representative of the embodiments and that the list is not intended to represent a closed list exclusive of further embodiments. Such wording may include “e.g., “etc.”, “such as”, “for example”, “and so forth”, “and the like”, etc., and other wording as will be apparent from the surrounding context.

No element, act, or instruction used in the description of the present application should be construed as critical or essential to the invention unless explicitly described as such. Also, as used herein, the article “a” is intended to include one or more items. Where only one item is intended, the term “one” or similar language is used. Further, the terms “any of” followed by a listing of a plurality of items and/or a plurality of categories of items, as used herein, are intended to include “any of,” “any combination of,” “any multiple of,” and/or “any combination of multiples of” the items and/or the categories of items, individually or in conjunction with other items and/or other categories of items. Embodiments of the inventions set forth herein can be modified by one skilled in the art to achieve the benefit of the teachings as presented in the above descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Moreover, the claims should not be read as limited to the described order or elements unless stated to that effect. In addition, use of the term “means” in any claim is intended to invoke 35 U.S.C. §112, ¶6, and any claim without the word “means” is not so intended.

What is claimed is:

1. A method of sensing and removing subclinical precancerous cells from unblemished skin without any clinical evidence of skin damage wherein the unblemished skin is not chronically exposed to sun or UV radiations, the method comprising:
topically applying to skin of a patient a composition essentially comprising an immunomodulator in an amount effective to induce erythema, scaling, crusting, or combinations thereof.

2. The method of claim 1, wherein the subclinical precancerous cells are at a molecular stage and have not reached a detectable form.

3. The method of claim 1, wherein the immunomodulator agent is a TLR7 agonist.

4. The method of claim 3, wherein the TLR7 agonist agent consists essentially of Imiquimod.

5. The method of claim 1, wherein the composition is applied in a dosage in a range of 0.01 to 1 mg/cm.sup.2.

6. The method of claim 1, wherein the composition is administered in a cycle of therapy in the range of two to four weeks.

7. The method of claim 1, wherein the composition is administered once or twice daily during the cycle of therapy.

8. The method of claim 1, wherein the cycle of therapy is repeated at least once on a substantially annual basis.

9. The method of claim 1, wherein the composition further comprises one or more pharmaceutically acceptable carriers or excipients.

10. A method of inducing a favorable immune cell response in skin having subclinical skin damage, no previous actinic keratoses, and no previous nonmelanoma skin cancer, and where the skin is not being treated for fine lines or clinical wrinkles, the method comprising:
topically administering to the skin an effective amount of a composition comprising an immune cell response agent during a cycle of therapy that is in the range of two to four weeks; and
observing the skin for the response;
wherein the composition is administered once or twice daily during the cycle of therapy, which is repeated at least once on a substantially annual basis.

11. The method of claim 10, wherein the composition is administered in a cycle of therapy in the range of two to four weeks.

12. The method of claim 10, wherein the composition is administered once or twice daily during the cycle of therapy.

13. The method of claim 10, wherein the cycle of therapy is repeated at least once on a substantially annual basis.

14. The method of claim 10, wherein the TLR7 agonist agent comprises Imiquimod.

15. The method of claim 10, wherein the composition comprises Imiquimod in an amount in the range of 0.25 to 10% by weight.

16. The method of claim 15, wherein the composition comprises Imiquimod in an amount in the range of 0.5 to 5% by weight.

17. A method of early diagnosis of actinic keratosis comprising activating the immune response against subclinical cell damage destined to develop actinic keratosis, the method comprising: topically administering to the skin of the patient a composition essentially comprising Imiquimod.

18. A method of claim 17, wherein the patient has not been chronically exposed to the sun or UV radiations.

19. A method of claim 18, wherein the patient is between twenty (20) and fifty-nine (59) years of age.

20. A method of preventing actinic keratosis comprising topically administering to unblemished skin, not being treated for AK, fine lines or clinical wrinkles, an amount of an immune cell response agent effective to induce death of sub-clinically damaged cells in the unblemished skin, wherein death of the sub-clinically damaged cells is visible as erythema, scaling and/or crusting.
